(19) United States
(12) Patent Application Publication
(10) Pub. No.: US 2012/0209249 A1
(43) Pub. Date: Aug. 16, 2012

(54) Injection Arrangement for a Flowable Drug

(75) Inventors: Nils Basso, Frankfurt am Main (DE); Thomas Nagel, Tharandt (DE); Rene Richter, Tharandt (DE); Robert Witt, Dresden (DE)

(73) Assignee: SANOFI-AVENTIS DEUTSCHLAND GMBH, Frankfurt am Main (DE)

(21) Appl. No.: 13/377,155

(22) PCT Filed: Jun. 18, 2010
(86) PCT No.: PCT/EP10/58615

§ 371 Date: Apr. 15, 2012

(30) Foreign Application Priority Data
Jun. 19, 2009 (EP) 09008046.6

(51) Int. Cl. A61M 5/30; A61M 5/20

(52) U.S. Cl. 604/506; 604/151; 604/70

(57) ABSTRACT

The invention refers to an injection arrangement (1) comprising a replaceable container cartridge (2) for a flowable drug (5), the container cartridge (2) comprising a deformable bag (4) arranged inside and sealed against a rigid outer casing (3), the deformable bag (4) being arranged for holding the flowable drug (5) and in fluid communication with an outlet (6), the rigid outer casing (3) fillable by a displacement fluid (7) for displacing the deformable bag (4), the injection arrangement (1) further comprising a reservoir (8) for the displacement fluid (7) and a pump (9) for pumping the displacement fluid (7) from the reservoir (8) into the rigid outer casing (3) and/or from the rigid outer casing (3) into the reservoir (8) via a fluid channel (10).

The invention refers to an injection arrangement for delivering a flowable drug.

Medicament delivery devices in general comprise a reservoir for the medicament and means for displacing the medicament from the reservoir, usually in the shape of a plunger.

Medicament delivery devices of this type are restricted to a generally cylindrical form of the reservoir. Furthermore the device with the filled reservoir is about twice as long as the reservoir alone due to the length of the plunger.

U.S. Pat. No. 5,527,288 discloses a reservoir in the form of an expansible-contractible chamber arranged within a housing member, the interior of the housing including a flexible liquid-impermeable membrane defining a first expansible-contractible chamber between it and a first non-deformable section of the housing, and a second expansible-contractible chamber defined between it and a second non-deformable section of the housing. The first chamber serves as a reservoir for receiving the drug to be delivered, whereas the second chamber typically serves as a gas-pumping chamber for controlling the delivery of the drug from the reservoir.

EP 1 450 882 B1 discloses a portable delivery device, pressurised by a pumped liquid and a cartridge therefore. The cartridge comprises a variable-volume drug reservoir having an outlet means and being adapted for containing a predefined volume of a flowable drug.

It is an object of the invention to provide an improved injection arrangement for delivering a flowable drug.

The object is achieved by an injection arrangement according to claim 1.

Advantageous embodiments are given in the dependent claims.

An injection arrangement according to the invention comprises a replaceable container cartridge for a flowable drug. The container cartridge comprises a deformable bag arranged inside and sealed against a rigid outer casing. The deformable bag is arranged for holding the flowable drug and is in fluid communication with an outlet for delivering the flowable drug to a human or an animal. The rigid outer casing is fillable by a displacement fluid for displacing the deformable bag. The injection arrangement further comprises a reservoir for the displacement fluid and a pump for pumping the displacement fluid from the reservoir into the rigid outer casing and/or from the rigid outer casing into the reservoir via a fluid channel.

As compared to conventional injection arrangements the injection arrangement according to the invention avoids the friction associated with bungs and plungers. Furthermore the dead volume is considerably smaller since the volume inside the deformable bag can virtually be reduced to zero whereas in conventional injection arrangements there is usually some residual drug even with after fully advancing the bung due to manufacturing tolerances. Except for the cartridge the injection arrangement according to the invention is reusable since the flowable drug does not get in contact with the pump or the reservoir. The cartridge may easily be replaced.

The injection arrangement can either be a stationary or a portable device such as an Insulin pen.

Preferably the pump may be motor driven.

The outlet can comprise a hollow needle or a jet nozzle.

The displacement fluid may be a liquid, for example oil and/or water. The liquid may have a low viscosity in order to reduce friction in narrow fluid passages. As liquids are almost incompressible, a high dosing accuracy of the flowable drug or medicament may be achieved.

In a preferred embodiment the deformable bag is attached at an end of the rigid outer casing opposite the outlet in order to ensure a precise folding of the deformable bag. E.g. in an elongate rigid outer casing the deformable bag is held at a front end in the region of the outlet where it is sealed against the rigid outer casing and at a back end opposite the outlet.

The deformable bag may be inserted into the rigid outer casing through the open outlet end of the rigid outer casing. The deformable bag may be attached to a closure member for sealing the open outlet end, wherein the closure member may have a fluid passage for allowing fluid communication between the deformable bag and the outlet.

The closure member may be glued or welded or crimped onto the open outlet end in order to seal the interior of the rigid outer casing.

The deformable bag may comprise silicone or another flexible material.

In an example embodiment, the pump is a piston pump. The pump may comprise an electric motor and a transmission to translate the rotational movement of the motor into a translational movement of the piston. The pump may further comprise a gear to adapt the number of rotations of the motor to a defined displacement of the piston. By using a piston pump, the amount of displacement fluid, and thus the amount of the drug or medicament, may be proportional to the number of piston movements. The number of piston movements may further relate to the number of motor rotations. Thus, based at least in part on the number of motor rotations and/or piston movements, the amount of the displacement fluid pumped from the reservoir into the rigid outer casing may be determined. Therefore, by controlling and/or monitoring the number of motor rotations, or by controlling and/or monitoring the number of piston movements, the amount of expelled medicament can be determined.

In a preferred embodiment a flux sensor is arranged between the pump and the rigid outer casing. This allows for setting and controlling a dose of the flowable drug to be injected by measuring the flux of the displacement fluid without having to get the flux sensor in contact with the drug. The volume of the displacement fluid pumped into the rigid outer casing is the same as the volume of the delivered drug as long as the displacement fluid is a liquid rather than a gas, since liquids are virtually incompressible as opposed to gases. However, a gas may also be employed as the displacement fluid.

The reservoir may be integrated with the pump in a pump module. The pump module may be reused since it does not get in contact with the drug. The reservoir may be refillable in order to be reused. In this case the container cartridge is removed from the injection arrangement after use with displacement fluid still inside the rigid outer casing. In another embodiment the displacement fluid may be pumped back from the rigid outer casing into the reservoir after injection so the reservoir does not need to be refilled.

The injection arrangement may be operated in an inverse mode by pumping the displacement liquid out of the rigid outer casing into the reservoir thereby creating a vacuum in the deformable bag so the flowable drug or another flowable substance, e.g. blood may be sucked through the outlet into the deformable bag.

The injection arrangement may be advantageously used for delivering a flowable drug of the group Insulin, Heparin, Lovenox, human growth hormones, peptide hormones, analgetics and vaccines.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

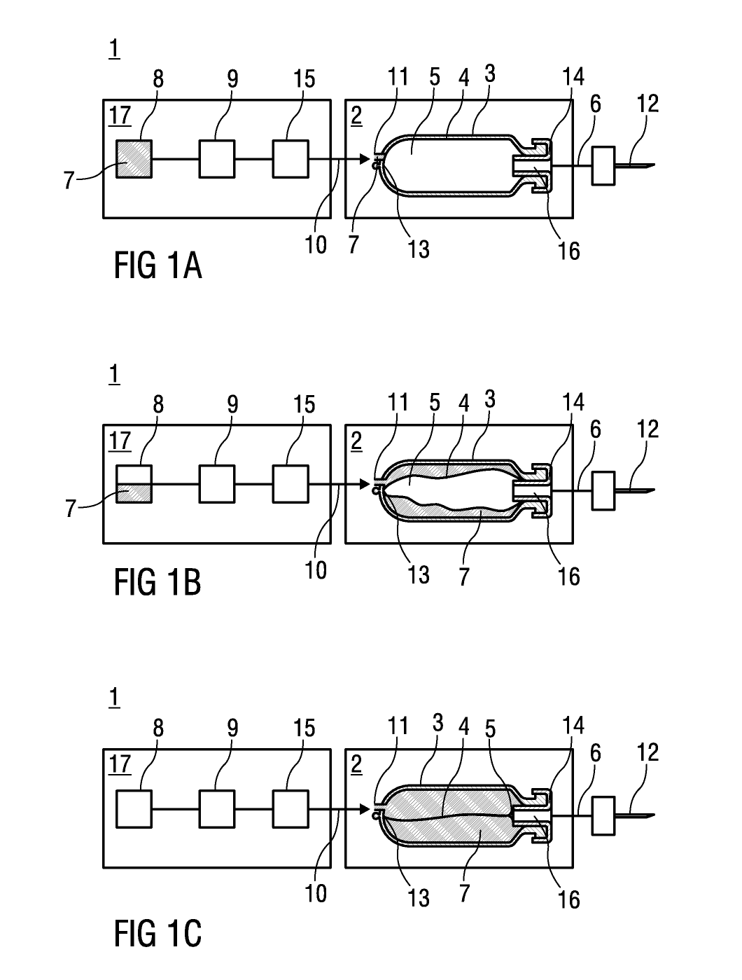

FIG. 1 is a schematic view of an injection arrangement with a container cartridge shown in a longitudinal section.

FIG. 1 shows a schematic view of an injection arrangement 1 with a replaceable container cartridge 2 shown in a longitudinal section. The container cartridge 2 comprises a rigid outer casing 3 and a deformable bag 4 arranged inside and sealed against the rigid outer casing 3. The deformable bag 4 holds a flowable drug 5 and is in fluid communication with an outlet 6 for delivering the flowable drug 5 to a human or an animal. The rigid outer casing 3 is fillable by a displacement fluid 7 for displacing the deformable bag 4 and thus the flowable drug 5 inside. The injection arrangement 1 further comprises a reservoir 8 for the displacement fluid 7 and a pump 9 for pumping the displacement fluid 7 from the reservoir 8 into the rigid outer casing 3 and/or from the rigid outer casing 3 into the reservoir 8 via a fluid channel 10 connected to the rigid outer casing 3 at an opening 11 thereof.

FIG. 1a shows the injection arrangement 1 in an initial state, where the deformable bag 4 with the flowable drug 5 essentially fills the entire volume of the rigid outer casing 3.

FIG. 1b shows the injection arrangement 1 in a state with the rigid outer casing 3 partially filled by the displacement fluid 7 thus squeezing or displacing the deformable bag 4 and the flowable drug 5 inside and forcing the flowable drug 5 out of the outlet 6 in order to inject it into a human or an animal. The injection may be stopped at this point if the required dose of the drug 5 has been injected without emptying the whole content of the deformable bag 4.

However, as shown in FIG. 1c, the whole content of the deformable bag 4 may be forced out of the outlet by entirely filling the rigid outer casing 3 with the displacement fluid 7 thereby fully squeezing the deformable bag 4.

The injection arrangement 1 can either be a stationary or a portable device such as an Insulin pen.

Preferably the pump 9 may be motor driven.

The outlet 6 can comprise a hollow needle 12 or a jet nozzle (not shown).

The displacement fluid 7 may be a liquid with a low viscosity such as oil and/or water. A gas may also be employed as the displacement fluid 7.

The deformable bag 4 is attached at an end of the rigid outer casing 3 opposite the outlet 6. In an elongate rigid outer casing 3 as shown in the figures the deformable bag 4 is held at a front end in the region of the outlet 6 where it is sealed against the rigid outer casing 3 and at a back end 13 opposite the outlet 6.

The deformable bag 4 may be inserted into the rigid outer casing 3 through the open outlet/front end of the rigid outer casing 3. The deformable bag 4 may be attached to a closure member 14 for sealing the open outlet/front end, wherein the closure member 14 may have a fluid passage 16 for allowing fluid communication between the deformable bag 4 and the outlet 6.

The closure member 14 may be glued or welded or crimped onto the open outlet/front end in order to seal the interior of the rigid outer casing 3.

The deformable bag 4 may comprise silicone or another flexible material.

A flux sensor 15 may be arranged between the pump 9 and the rigid outer casing 3. This allows for setting and controlling a dose of the flowable drug 5 to be injected by measuring the flux of the displacement fluid 7.

By measuring the amount of displacement fluid 7 with the flux sensor 15, the amount of drug or medicament 5 which is expelled may be determined without direct contact of the flux sensor 15 with the drug or medicament.

The reservoir 8 may be integrated with the pump 9 in a pump module 17. The pump module 17 may be reused. The reservoir 8 may be refillable. In this case the container cartridge 2 is removed from the injection arrangement 1 after use with the displacement fluid 7 still inside the rigid outer casing 3. In another embodiment the displacement fluid 7 may be pumped back from the rigid outer casing 3 into the reservoir 8 after injection so the reservoir 8 does not need to be refilled.

The injection arrangement 1 may be operated in an inverse mode by pumping the displacement liquid 7 out of the rigid outer casing 3 into the reservoir 8 thereby creating a vacuum in the deformable bag 4 so the flowable drug 5 or another flowable substance, e.g. blood may be sucked through the outlet 6 into the deformable bag 4. In this inverse mode the initial state is essentially the one shown in FIG. 1c.

The injection arrangement 1 may be advantageously used for delivering a flowable drug 5 of the group Insulin, Heparin, Lovenox, human growth hormones, peptide hormones, analgetics and vaccines.

The term “drug” or “medicament”, as used herein, may also mean a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-PheIle-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37
Exendin-4(1-39)-NH2,
H-(Lys)5-des Pro36, des Pro37
Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28]
Exendin-4(1-39),
des Pro36 [Met(O)14 Trp(O2)25, IsoAsp28]
Exendin-4(1-39);
or
des Pro36 [Asp28/ Exendin-4(1-39),
des Pro36 [IsoAsp28/ Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(02)25, Asp28]
Exendin-4(1-39),
des Pro36 [Met(O)14 Trp(02)25, IsoAsp28]
Exendin-4(1-39),

wherein the group -Lys6-NH2 may be bound to the C-terminus of the Exendin-4 derivative;
or an Exendin-4 derivative of the sequence

H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-
NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-
NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-
39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-
(Lys)6-NH2,
H-Asn-(Glu)5-des Pro36, Pro37, Pro38 [Met(O)14, Trp(O2)25, Asp28] Exendin-4(1-
39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### LIST OF REFERENCES

1 injection arrangement
2 container cartridge
3 rigid outer casing
4 deformable bag
5 flowable drug
6 outlet
7 displacement fluid
8 reservoir
9 pump
10 fluid channel
11 opening
12 hollow needle
13 back end
14 closure member
15 flux sensor
16 fluid passage
17 pump module

1. Injection arrangement comprising a replaceable container cartridge for a flowable drug, the container cartridge comprising a deformable bag arranged inside and sealed against a rigid outer casing, the deformable bag being arranged for holding the flowable drug and in fluid communication with an outlet, the rigid outer casing fillable by a displacement fluid for displacing the deformable bag the injection arrangement further comprising a reservoir holding the displacement fluid and a pump for pumping the displacement fluid from the reservoir into the rigid outer casing and/or from the rigid outer casing into the reservoir via a fluid channel, wherein the displacement fluid is a liquid.

2. Injection arrangement according to claim 1, characterized in that the outlet comprises a hollow needle.

3. Injection arrangement according to claim 1, characterized in that the outlet comprises a jet nozzle.

4. Injection arrangement according to claim 1, characterized in that the displacement fluid comprises oil and/or water.

5. Injection arrangement according to claim 1, characterized in that the deformable bag is attached at an end of the rigid outer casing opposite the outlet.

6. Injection arrangement according to claim 1, characterized in that the deformable bag is inserted into the rigid outer casing through an open outlet end of the rigid outer casing, wherein the deformable bag is attached to a closure member for sealing the open outlet end, the closure member having a fluid passage for allowing fluid communication between the deformable bag and the outlet.

7. Injection arrangement according to claim 6, characterized in that the closure member is glued or welded or crimped onto the open outlet end.

8. Injection arrangement according to claim 1, characterized in that the deformable bag comprises silicone.

9. Injection arrangement according to claim 1, characterized in that the pump is a piston pump comprising an electric motor wherein an amount of the displacement fluid pumped from the reservoir into the rigid outer casing may be determined based at least in part on the number of motor rotations.

10. Injection arrangement according to claim 1, characterized in that a flux sensor is arranged between the pump and the rigid outer casing.

11. Injection arrangement according to claim 1, characterized in that the reservoir is integrated with the pump in a pump module.

12. Injection arrangement according to claim 1, characterized in that the reservoir is refillable.

13. Use of an injection arrangement according to claim 1 for delivering a flowable drug of the group Insulin, Heparin, Lovenox, human growth hormones, peptide hormones, analgetics and vaccines.

14. Method for operating an injection arrangement, the injection arrangement comprising a replaceable container cartridge for a flowable drug, the container cartridge comprising a deformable bag arranged inside and sealed against a rigid outer casing, the deformable bag being arranged for holding the flowable drug and in fluid communication with an outlet, the rigid outer casing fillable by a displacement fluid for displacing the deformable bag, the injection arrangement further comprising a reservoir for the displacement fluid and a pump, the method comprising the step of pumping the displacement fluid from the reservoir into the rigid outer casing and/or from the rigid outer casing into the reservoir via a fluid channel, characterized in that the displacement fluid used is a liquid.

15. Method according to claim 14, characterized in that the displacement fluid comprises oil and/or water.

16. Method according to claim 14, characterized in that a flux sensor arranged between the pump (9) and the rigid outer casing is used for setting and controlling a dose of the flowable drug to be injected by measuring the flux of the displacement fluid.
